(19) United States
(12) Patent Application Publication
(10) Pub. No.: US 2024/0374413 A1
(43) Pub. Date: Nov. 14, 2024

(54) A MENSTRUAL COLLECTING DEVICE AND APPLICATOR

(71) Applicant: Fillow, Inc., Highland Park, IL (US)

(72) Inventors: Gregory James Foster, Chicago, IL (US); Robert Warren Sheldon, Winnetka, IL (US); Camryn Josie Ross, Highland Park, IL (US); Heekyoung Woo, Cambridge, MS (US); Seoyeon Choi, Seongnam-si (KR); Soyee Kim, San Diego, CA (US)

(21) Appl. No.: 18/686,556

(22) PCT Filed: Aug. 31, 2022
(86) PCT No.: PCT/US2022/042182

§ 371 Date: Feb. 26, 2024

Related U.S. Application Data
(60) Provisional application No. 63/239,460, filed on Sep. 1, 2021.

(51) Int. Cl. A61F 5/455

(52) U.S. Cl. CPC A61F 5/4553

(57) ABSTRACT

In one aspect, this disclosure relates to a menstrual collecting device as described in this disclosure. In another aspect, this disclosure relates to an applicator that aids the positioning of the menstrual collecting device in a subject. The disclosure further relates to a system comprising at least one applicator and one or more menstrual collecting devices which may be pre-loaded into the applicator.

### CROSS-REFERENCE TO RELATED APPLICATIONS

This application claims the benefit of priority from U.S. provisional patent application 63/239,460 filed Sep. 1, 2021, the entire disclosure of which is herein incorporated by reference in its entirety.

### TECHNICAL FIELD

This disclosure relates to products for menstruating users, including a menstrual disc and applicator.

### BACKGROUND

Current solutions available in the menstrual market involve short-lasting absorption techniques, found in tampons and pads, or consist of products that are inconvenient to place intravaginally to collect menses in menstrual cups and discs.

Cotton-based products' highly absorbent design can encourage bacterial growth and the risk of developing (1) Toxic Shock Syndrome (TSS). Menstrual cups and discs' popularity is limited due to its inconvenient usage. Factors that prohibit such popular use of these menstrual products include but are not limited to: (2) messy contact during insertion and removal, (3) difficulty in proper positioning, (4) difficulty in removal, and (5) leakage during usage.

Cotton-based products, such as pads and tampons, utilize absorption to take in menses and prevent it from leaking. The downside to these products is that oftentimes, a user's flow is heavy enough to the extent that the product can no longer absorb the menses, so leaking ensues. These products' highly porous materials are a medium that encourages bacterial growth, therefore enhancing the risk of developing Toxic Shock Syndrome.

Menstrual cups do not absorb menses, rather they hold and collect menses within the vaginal canal. The cup is designed to open up inside vagina and suction seal to the vaginal walls, which keeps it in place. A menstrual cup is inserted usually without the use of an applicator, meaning users manually place and open the cup in their vaginal canals. Similarly, during removal, users have to manually unseal the product to pull it out of their vaginal canals. Though the product is often made of silicone and does not utilize absorption, the task of manually inserting and removing the cup themselves is often seen as an uncomfortable, difficult, and messy task for users.

Menstrual discs are a disc-shaped device that is placed within the fornix and underneath the cervix in order to collect menses. Menstrual discs often require manual insertion and removal by the users, which pose difficulty due to disc's deeper placement in the body.

Thus, there remains the need for products that would improve menstruators' daily activities during their menstruation cycles.

### SUMMARY

This disclosure helps in addressing this and other needs and provides 1) a menstrual collecting device suitable for efficient collecting of menses to assist menstruators' daily activities during their menstruation cycles, 2) applicators for positioning the menstrual collecting device; and 3) systems containing one or more of these products.

In one aspect, the disclosure provides the menstrual collecting device which may be referred in this disclosure as a menstrual disc which may be reusable in some embodiments, or it may be disposable in other embodiments.

The menstrual collecting device may comprise a collection bag having a rim, the collection bag created by a wall enclosing a chamber having a volume for collecting menstrual discharge, the collection bag having a length between a bottom surface and a front surface, the collection bag having an opening located on the front surface of the collection bag, the rim surrounding the perimeter of the opening and having a volume for collecting menstrual discharge, and a pull string or a pull tab being attached to the rim or the wall below the rim.

In some embodiments, the rim may be defined further by one or more of the following features:
a) the rim is adjustable, and the adjustable rim includes an expandable/contractible feature,
b) the rim is a co-molded ridged material to create a spring, comprises opposing indents, is a spiral molded with variable wall thicknesses, is a spring coil molded with variable wall thicknesses, comprises a “C” spring, and/or comprises any other suitable collapsing feature;
c) the rim is substantially circular, elliptical, or any other suitable shape;
d) the rim is manufactured by a method which includes injection molding, co-molding and/or compression molding; and/or
e) the rim is concave.

In some embodiments, the menstrual collecting device according to this disclosure may further comprise a return wall positioned over a portion of the opening of the menstrual collecting device, the return wall being attached to the rim and/or to the wall of the menstrual collecting device. If the return wall is present, then in some preferred embodiments, the return wall may be defined further by one or more of the following features:
a) the return wall is angled downwards transitioning into the opening;
b) the opening is located at the center or side of the rim;
c) the opening takes the shape of a circle, a hexagon, a water droplet, a slit, an “x”, or any other suitable shape for collection of menstrual discharge in the collection bag;
d) the return wall's function is to withhold collected vaginal discharges or menses from escaping; and/or
e) the opening can be closed or sealed through different mechanisms involving folding, sealing, and/or covering the return wall for ease of disposal.

In some preferred embodiments of the menstrual collecting device according to this disclosure, the collection bag may be defined further by one or more of the following features:
a) some or all parts of the collection bag have different wall thickness to prevent sudden spillage upon the user's activity;
b) the collection bag is contoured to prevent spillage; and/or
c) the collection bag is in the shape of a semi-sphere, a spherical ellipsoid, the frustum of a cone, or a semi sphere that is flattened near the bottom.

Some preferred embodiments of the menstrual collecting device according to this disclosure include those, wherein the pull string or a pull tab is defined further by one or more of the following features:
a) the pull string is connected to the menstrual collecting device on or below the rim and serves to ease removal for the user;
b) the pull string or the pull tab is connected to the return wall;
c) the pull string or the pull tab may have texture for the user's grip; and/or
d) the pull string or the pull tab is wider at its point of attachment to the menstrual collecting device and tapers out.

In another aspect, this disclosure provides an applicator which may be reusable in some embodiments, or it may be disposable in other embodiments. The applicator is sized and shaped for positioning the menstrual collecting device according to this disclosure. The applicator may be composed of a barrel and a plunger, the plunger being insertable into the barrel, the barrel sized for housing the menstrual collecting device in a folded form and for inserting the menstrual collecting device under the cervix by pushing the plunger.

Preferably, the applicator may be further defined by one or more of the following features:
a) the barrel is straight, curved or angled;
b) the plunger is sized and shaped to mirror the shape of the barrel, and push the menstrual collecting device out of the applicator barrel and into the fornix;
c) the applicator has a clicking mechanism having a ring or flanges/fingers protruding inside the barrel and a portion of the external surface of the plunger having one or more notches;
d) the barrel contains two or more petal flaps at its distal end;
e) the barrel contains one or more grooves or ridges on the barrel wall for at least a portion of the barrel length, the grooves or ridges being located opposite or substantially opposite to each other;
f) the barrel is made of a material different than the plunger;
g) the petal flaps are made of a material which is softer and more flexible than the rest of the barrel body;
h) the plunger is hollow;
i) at its proximal end, the plunger ends with a stopper; and/or
j) the plunger contains one or more ridges located on the external surface of the plunger for at least a portion of the plunger length, the one or more ridges extending from the external surface of the plunger.

In yet another aspect, this disclosure relates to a system or a kit comprising at least one applicator according to this disclosure and one or more of the menstrual collecting devices according to this disclosure.

In yet further aspect, this disclosure relates to a method for manufacturing the menstrual collecting device, applicator or the system according to this disclosure, wherein the method may include one or more of the following: injection molding, co-molding, heat bonding/sealing/staking, radiofrequency sealing, ultrasonic/thermal impulse/radiofrequency welding, dip molding, thermoforming, vacuum forming, compression molding, and/or cutting.

In yet another aspect, this disclosure relates to a method for collecting menstrual discharge, the method comprising:
folding and inserting the menstrual collecting device into the applicator barrel of the applicator,
inserting the plunger into the barrel,
inserting the applicator loaded with the menstrual collecting device into the user's vagina,
pushing the plunger of the applicator to release the menstrual collecting device and ensure its correct positioning under the cervix,
removing the applicator,
collecting menstrual discharge with the menstrual collecting device, and
pulling the pull string or the pull tab of the menstrual collecting device in order to remove the menstrual collecting device from the vagina.

In further aspect, this disclosure relates to uses of the products according to this disclosure and methods of personal care during a menstrual cycle.

### BRIEF DESCRIPTION OF THE DRAWINGS

FIG. 1A depicts a front view of one embodiment of a menstrual collecting device according to this disclosure.

FIG. 1B depicts a side view of the embodiment of FIG. 1A.

FIG. 2A depicts a front view of an embodiment of a menstrual collecting device according to this disclosure.

FIG. 2B depicts a side view of the embodiment of FIG. 2A.

FIG. 3A depicts a front view of an embodiment of a menstrual collecting device according to this disclosure.

FIG. 3B depicts a side view of the embodiment of FIG. 3A.

FIG. 3C is a close up of the rim in FIG. 3A that shows one possible mechanism of achieving self-sizing fit.

FIG. 4A depicts a front view of an embodiment of a menstrual collecting device according to this disclosure.

FIG. 4B depicts a side view of the embodiment of FIG. 4A.

FIG. 5A depicts a front view of an embodiment of a menstrual collecting device according to this disclosure.

FIG. 5B depicts a side view of the embodiment of FIG. 5A.

FIG. 6A depicts a front view of one embodiment of a system according to this disclosure showing a menstrual collecting device according to this disclosure positioned in an applicator according to this disclosure.

FIG. 6B is a side view of the embodiment of FIG. 6A.

FIG. 6C depicts the menstrual collecting device being released from the applicator of FIG. 6A.

FIG. 6D is a side view of the embodiment shown in FIG. 6C.

FIG. 7 depicts a front view of an embodiment of a menstrual collecting device according to this disclosure.

FIG. 8A depicts a side view of an embodiment of a menstrual collecting device according to this disclosure.

FIG. 8B depicts a front view of the embodiment of FIG. 8A.

FIG. 9A depicts a side view of an embodiment of a menstrual collecting device according to this disclosure.

FIG. 9B depicts a front view of the embodiment of FIG. 9A.

FIG. 10 depicts a front view of an embodiment of a menstrual collecting device according to this disclosure.

FIG. 11 depicts a front view of an embodiment of a menstrual collecting device according to this disclosure.

FIG. 12A depicts a side view of an embodiment of a menstrual collecting device according to this disclosure.

FIG. 12B depicts a front view of the embodiment of FIG. 12A.

FIG. 12C is a cross-section along the 12C-12C axis of the menstrual collecting device shown in FIG. 12B.

FIG. 13A depicts one embodiment of an applicator according to this disclosure, including a plunger and a barrel.

FIG. 13B depicts a cross-sectional view along the long axis of the applicator of FIG. 13A with the plunger of FIG. 13A being assembled with the barrel of FIG. 13A.

FIG. 13C depicts a cross-sectional view along the long axis of the applicator of FIG. 13A with the plunger being fully inserted into the barrel.

FIG. 14A depicts a cross-sectional view along the long axis of the applicator of FIG. 13A, showing a portion of the plunger and a portion of the barrel, zooming onto a clicking mechanism in the barrel.

FIG. 14B is a perspective view of another embodiment of a barrel.

FIG. 14C depicts inside the barrel from its proximal end, showing a ring that assists the clicking mechanism.

### DETAILED DESCRIPTION

In one aspect, this disclosure provides a menstrual collecting device, and in particular a menstrual disc. Some embodiments include reusable menstrual discs, while other embodiments include disposable menstrual discs.

In another aspect, this disclosure relates to an applicator which can be used for placing a menstrual disc according to this disclosure into a user's body. In some embodiments, the applicator may be disposable. In other embodiments, the applicator may be reusable.

In yet a further aspect, this disclosure relates to a system or kit comprising one or more menstrual collecting devices according to this disclosure and at least one applicator according to this disclosure. The systems or kits may include disposable menstrual collecting devices and applicators and/or reusable menstrual collecting devices and applicators. Some systems or kits may include at least one component of the system or kit being reusable, e.g., the applicator, while the other component, e.g., the menstrual collecting device being disposable.

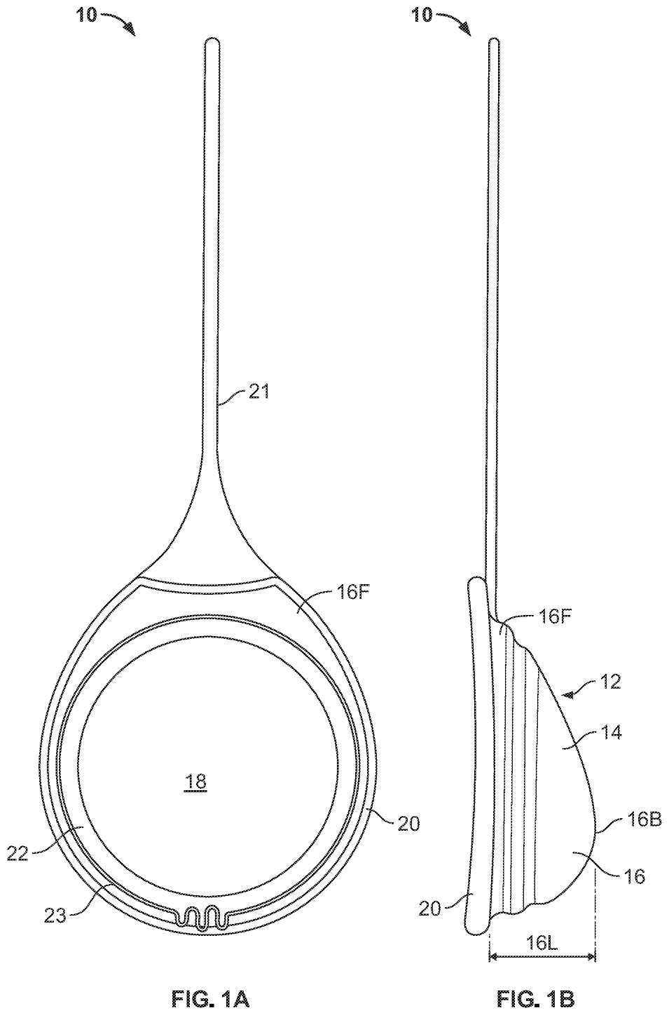

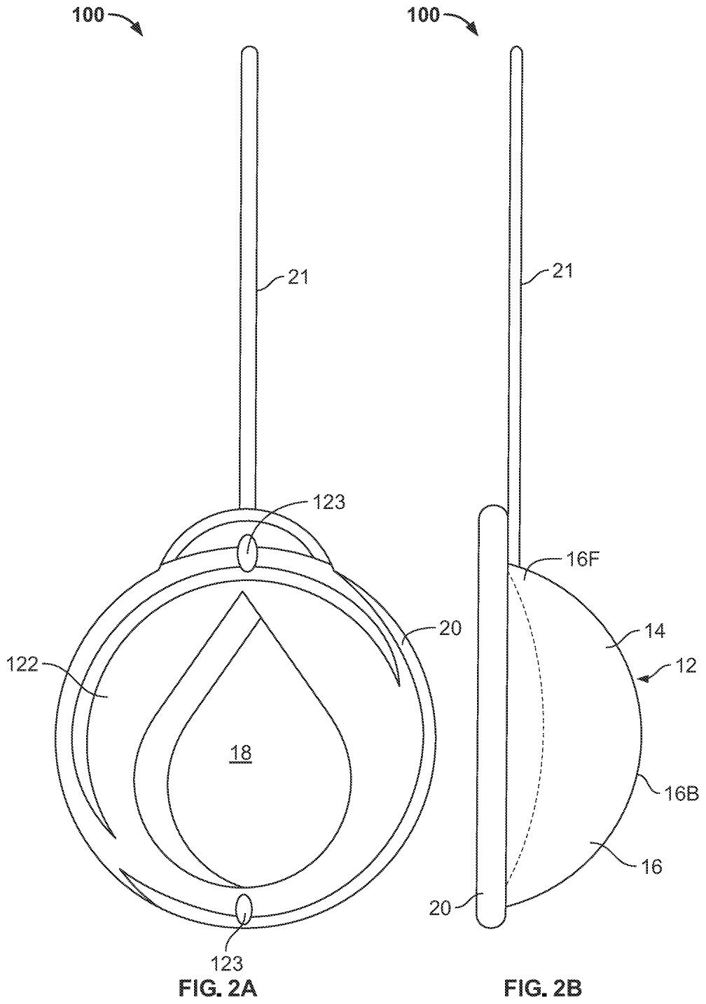

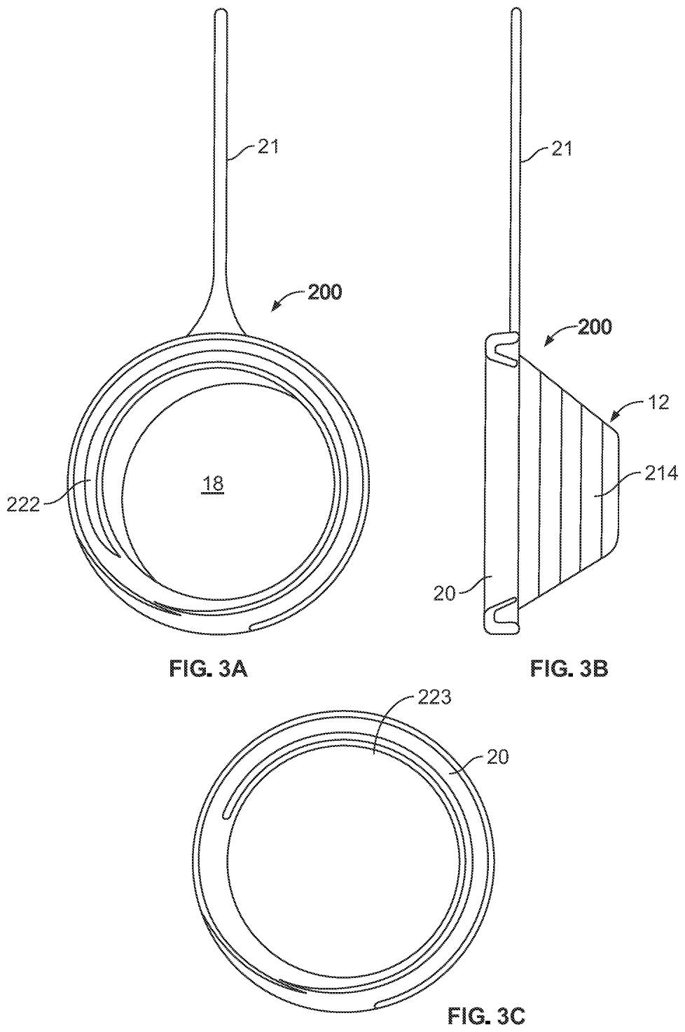

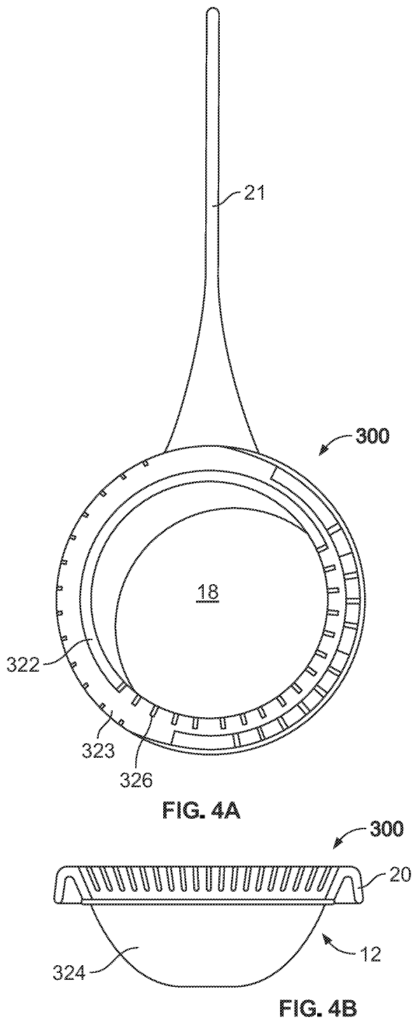

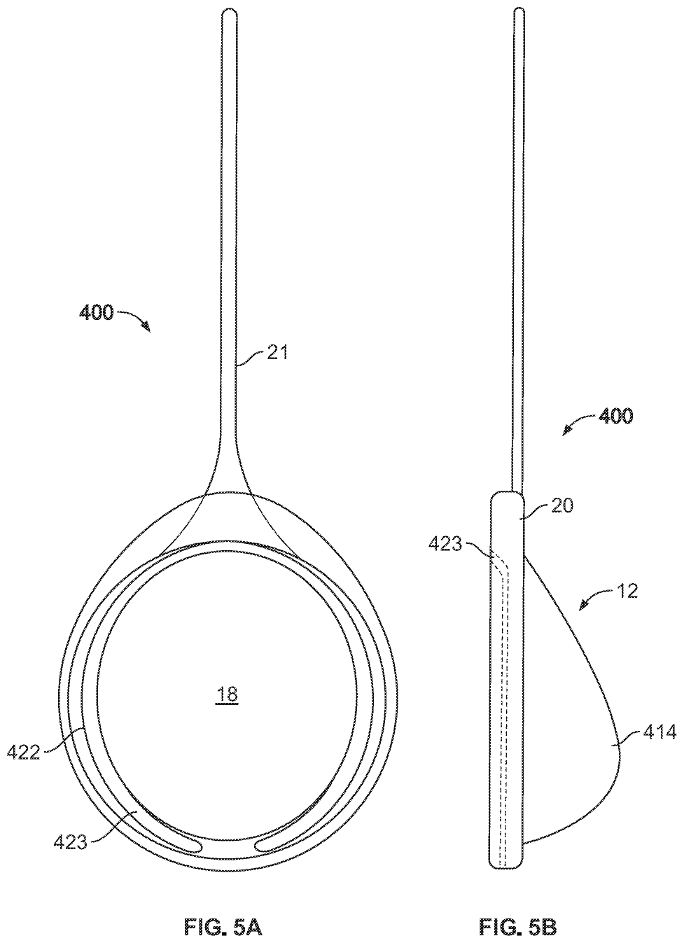

With reference to FIGS. 1A, 1B, 2A, 2B, 3A, 3B, 3C, 4A, 4B, 5A, 5B, 7, 8A, 8B, 9A, 9B, 10, 11, and FIGS. 12A, 12B and 12C, various embodiments of a menstrual collecting device will now be described in more detail. In some embodiments, a menstrual collecting device according to this disclosure, (generally 10, 100, 200, 300, 400, 500, 600, 700, 800, 900, or 1000), may have: a flexible collection bag (12 or 1010) created by a wall (14, 214, 324, 414 or 1014) enclosing a chamber (16) which may be bowl-like when holding a volume of liquid for collecting at least menstrual discharge, the chamber (16) having a height (16L or 1014L) between a bottom surface (16B or 1014B) and a front surface (16F or 1014F), the chamber having an opening (18, 918 or 1018) located on the front surface (16F or 1014F) of the collection bag (12 or 1010). The purpose of the collection bag (12 or 1010) is to collect and contain menstrual discharge.

In this disclosure, the “face surface” means the surface on which an opening (18, 918 or 1018) into a collection bag (chamber) is located. In this disclosure, the “bottom surface” is the surface opposite to the face surface. The collection bag (12 or 1010) has a volume between the front surface (16F or 1014F) and the bottom surface (16B or 1014B) for collecting menstrual discharge.

In this disclosure, the “internal surface,” e.g., the internal surface of the collection bag, barrel or plunger means the surface of the wall which is inside the chamber, collection bag, barrel or plunger. The “external surface,” e.g. the external surface of the collection bag, barrel or plunger, is the outside surface of the wall which is opposite to its internal surface.

In this disclosure, the same element, for example, element (21), may be included in different embodiments. In these different embodiments, the same element, e.g., element (21), is preferably denoted with the same reference number.

In this disclosure, the collection bag (12 or 1010) may be referred to as chamber or a bowl. In some embodiments, the collection bag (12) may be foldable and/or expandable in order to facilitate insertion, especially, a placement with an applicator. When unfolded and holding a volume of liquid, the collection bag (12 or 1010) in at least some embodiments may be a bowl or reservoir having a substantially curved wall (14 or 1014) in some embodiments and capable of containing a volume of menstrual discharge. The collection bag (12 or 1010) has a bottom surface (16B or 1014B) which may be at least partially flattened (214, 324), a front surface (16F or 1014F) and a height (16L or 1014L) between the bottom surface (12B or 1014B) and the front surface (12F or 1014F).

The collection bag (12 or 1010) functions as a reservoir for collecting menstrual discharge. Preferably, the collection bag (12 or 1010) may hold at least 20 ml, 30 ml, or more preferably at least 40 ml of liquid. The collection bag (12 or 1010) may be made of an elastic or semi-elastic material molded into a bowl-like structure.

Suitable dimensions for the collection bag (12 or 1010) include, but are not limited to, the height (16L or 1010L) in the range from about 10 mm to about 40 mm, and more preferably from about 15 mm to about 35 mm and/or a diameter of the front surface in the range from about 55 mm to about 75 mm, and more preferably from about 60 mm to about 70 mm.

Some or all parts of the collection bag (12 or 1010) may or may not have different wall thickness (14, 214, 324, 414 or 1014) to prevent sudden spillage upon the user's activity. The collection bag may or may not be contoured to prevent spillages. The collection bag may or may not be in the shape of a semi sphere, a spherical ellipsoid that may or may not be tilted towards a pull string (21), the frustum of a cone, a semi sphere that is flattened near the bottom, and any other suitable shape. This collection bag may or may not be heat staked to the adjustable rim, molded as a single injection part, etc.

In some embodiments, as shown for example, in wall embodiments (14) or (1014), the wall may be of substantially the same thickness from its bottom (16B or 1014B) to its front surface (16F or 1014F). However, in other embodiments, for example, as shown in an embodiment of (214), the wall may have variable wall thickness in order to improve the wall capacity to expand and contract as needed when in use. Yet, in another embodiment as shown in wall (324), the wall may contain ridges in order to improve the collection bag capacity to fit within the user's body.

The menstrual collecting device comprises a rim (20 or 1012). At its front surface (16F or 1014F), the wall (14 or 1014F) is connected to a rim (20 or 1012) located around the perimeter of the front surface (16F or 1014F) of the collection bag (12 or 1010) and attached to the wall (14, 214, 324, 414 or 1014) via heat staking for example or co-molded with the wall (14, 214, 324, 414 or 1014) of the collection bag (12 or 1010). In some embodiments, the rim (20 or 1012) and the collection bag (12 or 1010) may be manufactured together as one piece. This may be particularly preferred for embodiments in which a menstrual collecting device is reusable.

In other embodiments, preferably in embodiments with disposable menstrual collecting devices, the rim (20 or 1012) and the collection bag (12 or 1010) may be produced separately, and the rim (20 or 1012) is then attached to or near the face surface (14F or 1014F) of the wall (14 or 1014) of the collection bag (12 or 1010).

One of the functions for the rim (20 or 1012) is to ensure a better fit in order to prevent leakage while the device (10, 100, 200, 300, 400, 500, 600, 700, 800, 900 or 1000) is in use. Another function of the rim is to collect and contain menstrual discharge. The rim (20 or 1012) may be circular, elliptical, or in any other suitable shape.

In some embodiments, the rim (20 or 1012) may incorporate an expandable/contractible feature in order to produce an adjustable fit during the use.

The rim (20 or 1012) of the menstrual collecting device according to this disclosure may incorporate a spring/coil, or an expandable feature, that self-sizes the rim to the contour of the user's body upon insertion, providing a flexible range of rim sizes for a personalized, comfortable fit. Self-sizing is characterized by the rim having the capacity to remain in its most expanded state or shrink down to a smaller size based on the user's fornix size.

Some embodiments of the adjustable rim include, but are not limited to, such as a rim (20) being co-molded with ridged material to create a spring (23), a rim (20) having opposing indents (123), a spiral molded rim (20) with variable wall thicknesses (223, 214), a rim (20) having inner grooves (326) and outer grooves (323), a rim (20) with a spring coil molded with variable wall thicknesses (a combination of features 223 and 23), a rim (20) having a co-molded “C” spring (423), and/or any other suitable collapsing feature. The adjustable rim may be circular, elliptical, or any other suitable shape. Methods of manufacturing may or may not include injection molding, co-molding, compression molding, etc. The adjustable embodiments for the rim (20 or 1012) are suitable for every user's body, as the adjustable rim can shrink or expand as needed.

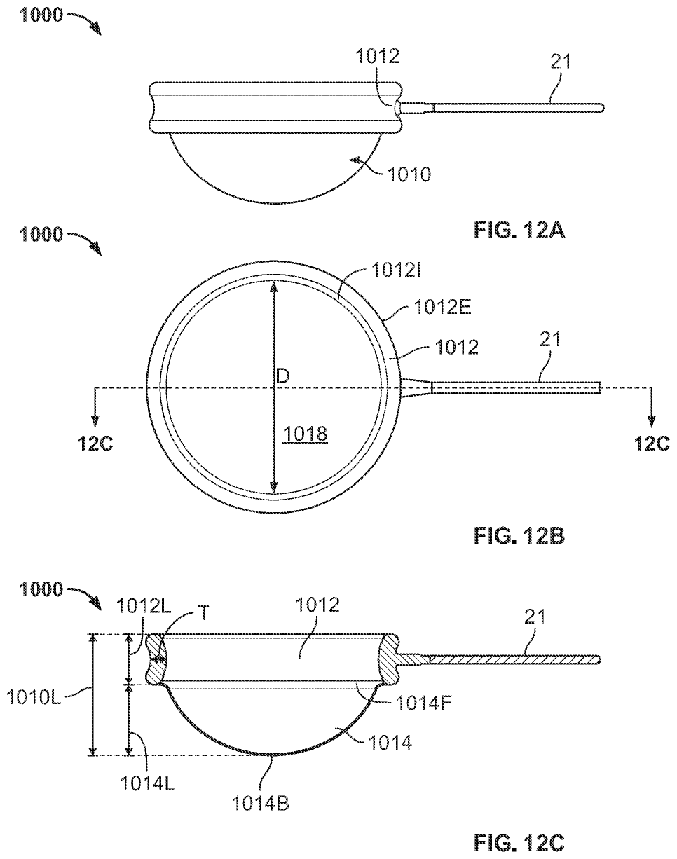

In some other embodiments, the rim (20 or 1012) may not include a separate expandable/contractible feature such as for example a spring and/or indents. In some embodiments, a rim, one embodiment of which (1012) is shown in FIG. 12A-12C, may be concave through its thickness (denoted as T in FIG. 12C), or at least partially concave or semi-concave at the outer edge which is opposite to the edge by which the rim (1012) is connected to the wall (1014) of the collection bag (1010). This feature improves the fit within the user's body.

The rim (20 or 1012) may be circular or substantially circular and self-sizing, having the capacity to remain in an expanded state or shrink down to a smaller size, based on the user's fornix size, providing a comfortable fit and minimizing leakage. In some embodiments, the rim (20 or 1012) may have a diameter (denoted as D in FIG. 12B) in the range from about 55 mm to about 75 mm, and more preferably from 60 mm to about 70 mm.

The wall of the rim (20 or 1012) can be made of any thickness (T), for example, in the range from about 2 mm to about 7 mm. The wall may be concave or semi-concave. The height (1012L) of the rim may be adjusted as needed, for example, from about 10 mm to about 20 mm. The rim (20 or 1012) has an external surface (denoted 1012E in FIG. 12B) and an internal surface (denoted 10121 in FIG. 12B). Some embodiments of the rim (20 or 1012) may incorporate a spring or a coil, while other embodiments do not contain a spring and/or coil. In order to improve the seal and prevent leakage, the rim (20 or 1012) may be shaped such that it facilitates the self-sizing function of the rim (20 or 1012). For example, the rim (20 or 1012) may be thicker at the edges than at its center and/or a perimeter of the rim center may be smaller than a perimeter of at least one of the two edges, resulting in a concave or a substantially concave shape, one embodiment of which is shown in FIG. 12C.

In some embodiments, a menstrual collecting device according to this disclosure may include a return wall (22, 122, 222, 322, 422, 522, 622, 722, 822 or 922). In other embodiments, for example as is shown the embodiment (1000) in FIGS. 12A-12C, the menstrual collecting device does not contain a return wall.

If present, the return wall (22, 122, 222, 322, 422, 522, 622, 722, 822 or 922) may be positioned over at least a portion of the front surface of the menstrual collecting device according to this disclosure. The return wall (22, 122, 222, 322, 422, 522, 622, 722, 822 or 922) may be attached to the rim (20) and/or to the wall (14, 214, 324 or 414). The return wall helps in retaining the collected vaginal discharge or menses collected within the device to avoid spillage during user's activities. The return wall (22, 122, 222, 322, 422, 522, 622, 722, 822 or 922) may or may not be angled downwards transitioning into the opening (18 or 918) that may or may not be at the center or side of the rim (20). The opening (18 or 918) may or may not take the shape of a circle, a hexagon, a water droplet, a slit, an “x”, and any other suitable shape. The return wall's function is to withhold collected vaginal discharges or menses from escaping during the user's activity. In some preferred embodiments, the return wall (122) may be concave or substantially concave, rounding inward toward the collection bag (12).

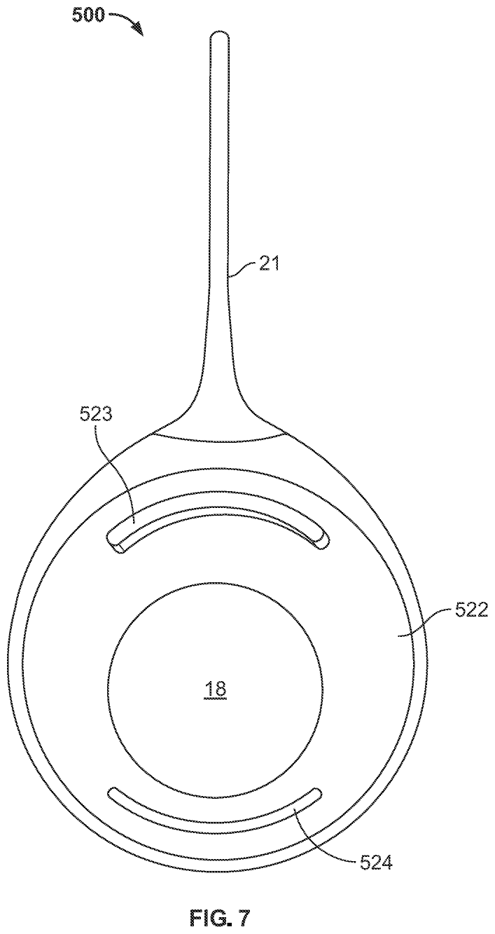

In some embodiments and as is shown in FIG. 7, the return wall (522) may contain at least one zip-lock raised feature (523) and at least one zip-lock recess (524) located on the external surface of the return wall (522), the zip-lock raised feature (523) and the zip-lock recess (524) being spaced from each other such that the opening (18) is positioned between the zip-lock raised feature (523) and the zip-lock recess (524), the zip-lock recess (524) having a depth and width such that the zip-lock raised feature (523) fits tightly into the recess (524) and the zip-lock raised feature (523) and the zip-lock recess (524) being spatially aligned with each other such that when the return wall (522) is folded with its external surface being inside the fold, the zip-lock raised feature (523) may be aligned at least partially with the zip-lock recess (524), fits into the recess (524) and zip-locks the menstrual collecting device. This feature may be helpful in convenient disposal of disposable menstrual collecting devices.

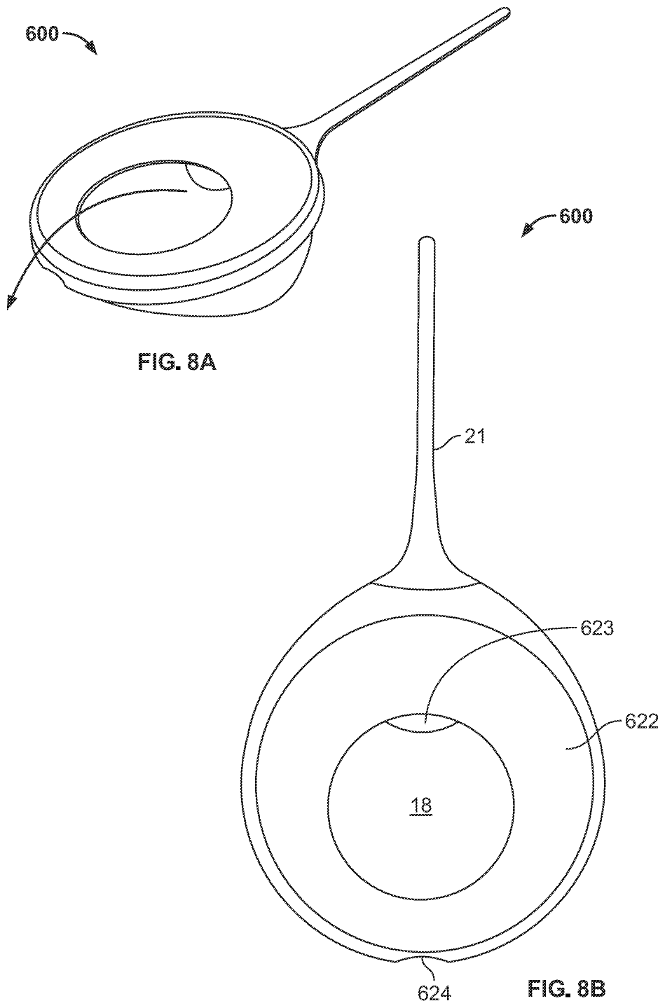

In some embodiments and as is shown in FIG. 8, the return wall (622) may contain at least one fold-in lock tab (623) attached to or near the free edge of the return wall (622). The free edge being the edge of the return wall (622) surrounding the opening (18). Preferably, the tab (623) is located on the internal surface of the return wall (622). The return wall (622) also includes at least one fold-in lock (624) which may be located on the return wall (622) or the rim (20) or the wall (14). The fold-in lock tab (623) and the fold-in lock (624) are spaced from each other such that the opening (18) is positioned between them. The fold-in lock tab (623) and the fold-in lock (624) are spatially aligned with each other such that when the return wall (622) is folded with its external surface being inside the fold, the fold-in lock tab (623) may be aligned at least partially with the fold-in lock tab (624) and the tab (623) fits into the lock (624) and locks the menstrual collecting device. This feature may be helpful after a disposable menstrual collecting device is getting disposed of and contains a volume of liquid.

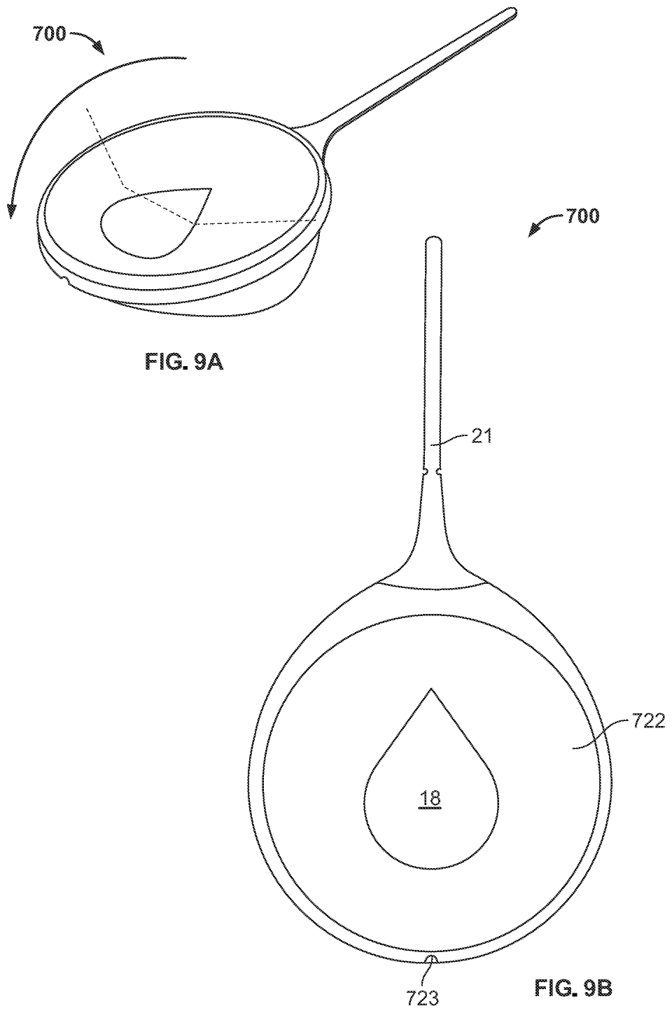

In alternative and in order to aid with a disposal of a menstrual collecting device containing a volume of liquid (menstrual discharge) in some embodiments and as is shown in FIGS. 9A and 9B, the rim (20) or return wall (722) may incorporate a string lock (723) which would fold the menstrual collecting device (700) by closing the external edges of the return wall (722) towards the opening (18) as shown in FIG. 9A when a user pulls a string (21). This feature may be helpful after a disposable menstrual collecting device is getting disposed of and contains a volume of liquid.

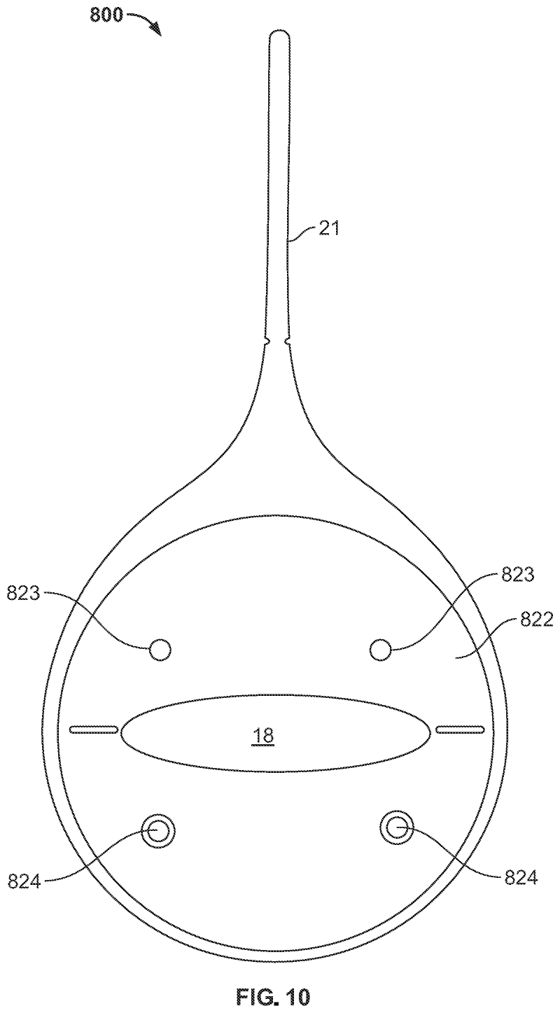

In some embodiments and as is shown in FIG. 10, the return wall (822) may contain one or more button-raised features (823) and complementary one or more button lock recesses (824) located on the external surface of the return wall (822). The button-raised features (823) and the button recesses (824) being spaced from each other such that the opening (18) is positioned between the button-raised features (823) and the button-lock recesses (824). The button-lock recess (824) has a depth and width such that the button raised feature (823) may fit snugly into the recess (824). The button-raised feature (823) and the button lock recess (824) are spatially aligned with each other on the surface of the return wall (822) such that when the return wall (822) is folded with its external surface being inside the fold, the button raised feature (823) may be aligned at least partially with the button-lock recess (824), fits into the recess (824) and locks the menstrual collecting device. This feature may be helpful for convenient disposal of a disposable menstrual collecting device that contains a volume of liquid.

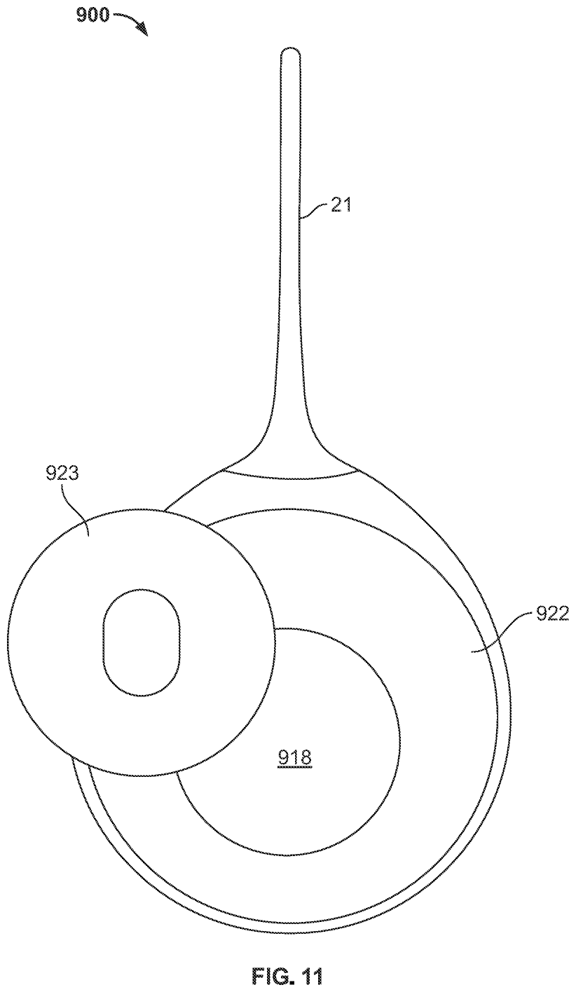

In some embodiments and as shown in FIG. 11, the menstrual collecting device (900) may include a removable lid (923) sized and shaped to close the opening (918) in the return wall (922). This feature may be helpful for convenient disposal of a disposable menstrual collecting device that contains a volume of liquid wherein a user removes the device (900) from her body and closes the device (900) by placing the lid (923) over the opening (918).

In some embodiments, the menstrual collecting devices according to this disclosure may contain a pull string (21) which may be in some embodiments replaced with a pull tab (not shown in drawings). In some other embodiments (not shown in drawings), a menstrual collecting device according to this disclosure does not contain a pull tab or a pull string.

When present, the pull string (21) or the pull tab may be attached to the rim (20 or 1012) and/or to the wall (14 or 1014) below the rim (20 or 1012). The pull string or the pull tab is connected to the menstrual collecting device on or below the rim and serves to ease removal for the user. The pull string may or may not have texture for the user's grip.

The pull string (21) or the pull tab in place of the pull string (21) is made of a flexible material and of the length that when in use, a portion of the pull string or the pull tab may extend from the user's body, allowing the user to remove the menstrual collecting device by pulling the pull string (21) or the pull tab. In some embodiments, the length (L) of the pull string (21) or the pull tab may be in the range from about 45 mm to about 75 mm, and preferably from about 55 mm to about 70 mm.

The pull tab is preferably wider than the pull string (21), for example, with the width (W) from about 10 mm to about 25 mm and flatter than a pull string, for example with the thickness of about 0.1 mm or 0.5 mm, helping in preventing skin irritation.

After collection is completed, the pull string (21) or the pull tab may aid in the removal of the device from underneath the cervix towards the opening of the vaginal canal.

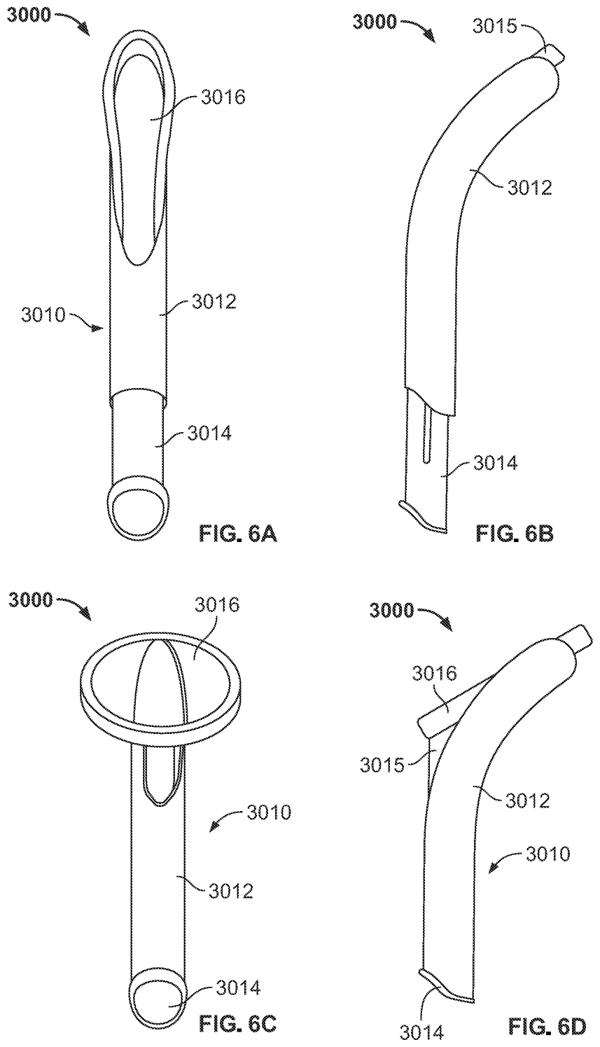

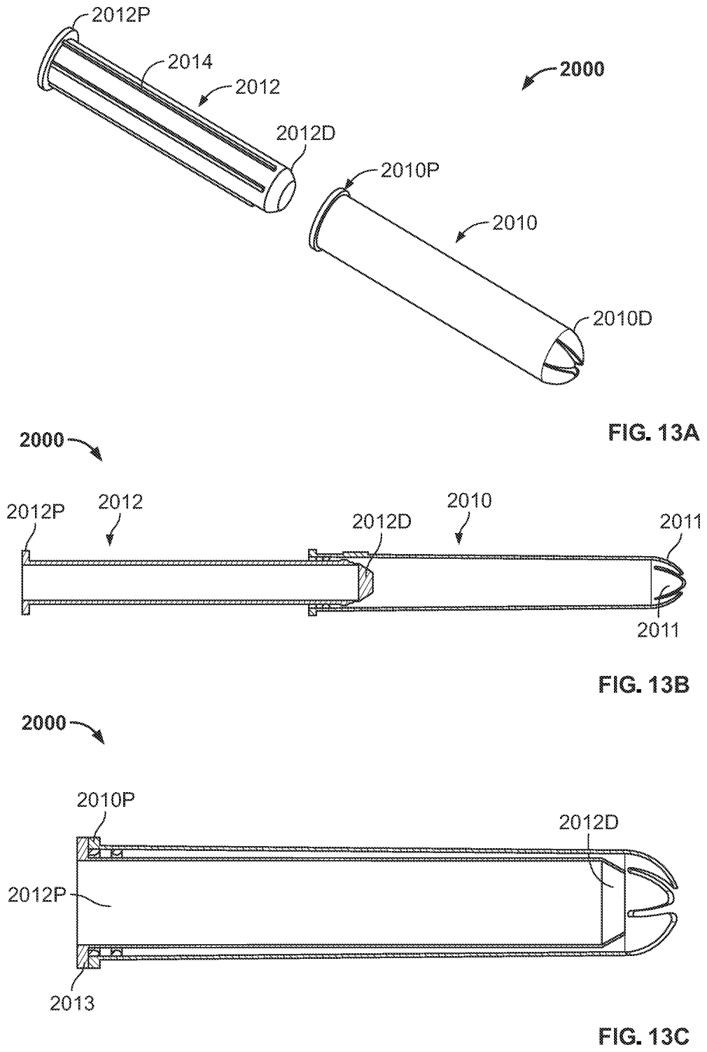

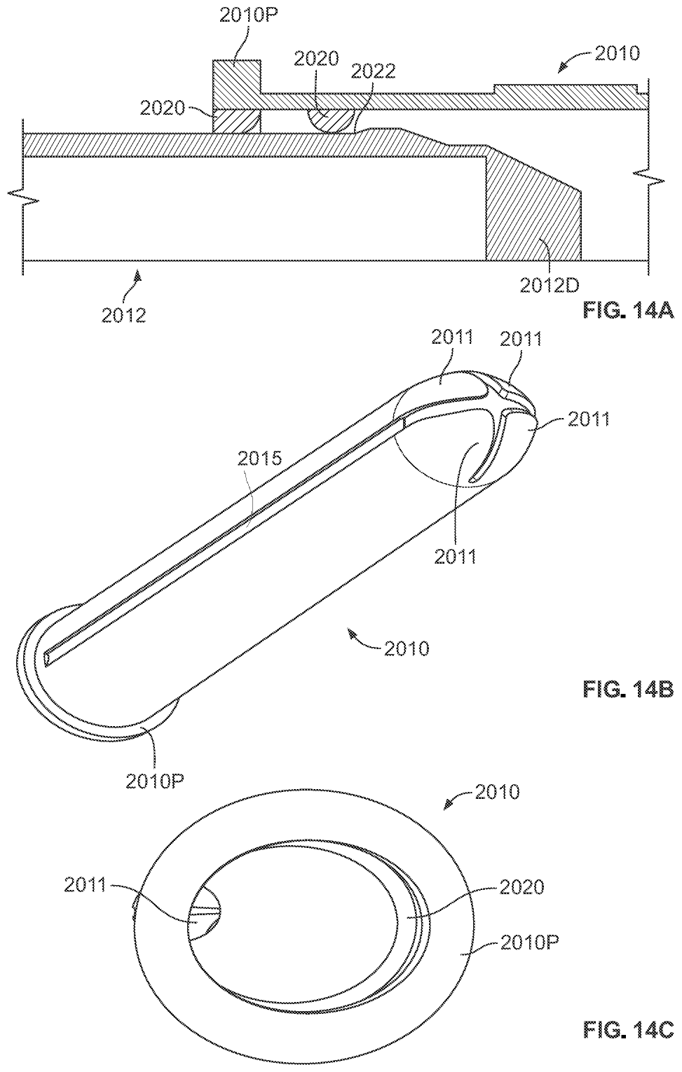

One of the technical advantages of the menstrual collecting devices according to this disclosure is that they can be placed by the user and correctly positioned with an applicator. In another aspect, this disclosure relates to an applicator, some embodiments of which are shown in FIGS. 13A, 13B, 13C, 14A, 14B, 14C, 6A, 6B, 6C and 6D. In some embodiments, an applicator generally (2000) (FIGS. 13A-14C) or (3010) (FIGS. 6A-6D), may comprise or consist of a plunger (2012 or 3014) insertable into a barrel (2010 or 3012).

In some preferred embodiments, the applicator (2000) may consist of a hollow applicator barrel which may be referred to simply as a barrel (2010 or 3012) and a plunger (2012 or 3014) insertable into the barrel (2010 or 3012). The barrel (2010 or 3012) may be straight, curved or angled. One embodiment of the straight barrel (2010) is shown in FIG. 13B. One embodiment of curved or angled barrel (3012) is shown in FIG. 6B. Preferably, the curvature is at the distal portion and away from the front surface of the barrel (3012). The distal portion is the portion which is inserted first during the use. The front surface is the surface which is aligned with the front surface of the menstrual collection device when in use.

The plunger (2012 or 3014) is designed to push the collecting device (10, 100, 200, 300, 400, 500, 600, 700, 800, 900, or 1000) out of the barrel (2010 or 3012). The plunger (2012 or 3014) may or may not be curved, depending upon the shape of the barrel. Preferably, if the barrel (3012) is curved, then the plunger (3014) is curved to mirror the curvature of the barrel and improve the fit of the plunger (3014) into the barrel (3012). In some preferred embodiments with reference to FIGS. 13A-14C, the barrel (2010) has a proximal end (2010P), a distal end (2010D) and a length between the distal end (2010D) and the proximal end (2010P). The barrel (2010) is hollow, encircling a hollow channel (bore) with an opening at the distal end (2010D) and an opening at the proximal end (2010P). As shown in FIG. 14B, the barrel (2010) may have one or more grooves (2015) or ridges (not shown in drawing) on the barrel wall, which serves for better user grip on the barrel (2010) as well as properly orienting the menstrual collecting device inside the barrel (2010) and inserting the menstrual collecting device. This is due to the menstrual collecting device having a distinguished opening/front surface and a bottom surface. The grooves (2015) or ridges (not shown in drawing) can extend anywhere from the distal end of the barrel (2010) all the way to the petal flaps (2011). In some preferred embodiments, the grooves (2015) or ridges may be located on the opposite sides of the barrel and end midway through the barrel (2010).

The plunger (2012) which has a distal end (2012D) and a proximal end (2012P) and a length between the distal end (2012D) and the proximal end (2012P) is sized and shaped to be insertable through the opening at the proximal end (2010P) of the barrel (2010) into the hollow channel of the barrel (2010), as shown for example, in FIG. 13B. The plunger (2010) is further sized and shaped such that the plunger (2012) is fit to move within the hollow channel of the barrel (2010) along the longitudinal axis 2010P/2010D of the barrel (2010). The plunger (2010) may be made hollow in at least some embodiments.

The distal end (2012D) of the plunger (2012) may be tapered as shown in FIG. 13B and/or the plunger may contain one or more prongs at its distal end.

At its proximal end (2012P), the plunger (2012) may end with a stopper (2013) which may rest on the wall of the proximal end (2010P) of the barrel (2010) after the plunger (2012) is fully inserted into the barrel (2010) as shown for example in FIG. 13C. The stopper (2013) prevents the plunger (2012) from completely sliding into the barrel (2010).

When in use, the plunger (2012 or 3014) pushes the menstrual collection device according to this disclosure, e.g., (3016) in FIGS. 6A-6D, out of the distal end (2010D or 3012D) of the applicator barrel (2010 or 3012) as shown for example in FIG. 6C, and further may push the menstrual collecting device (3016) into the fornix.

At its distal end (2010D), the barrel (2010) may have several petal flaps (2011). For example, three, four or more petal flaps (2011) as shown in FIG. 14B. The petal flap (2011) is a flap which is attached with its one edge to or near the distal end (2010D) of the barrel (2010). The opposite edge of the petal flap (2011) remains free and is not attached. Each petal flap (2011) is slightly curved toward the central long axis of the barrel (2010) and the free edge of the petal flap (2011) may be tapered. When in their closed position and as shown in FIG. 14B, the petal flaps (2011) hang over the distal opening (2010D) of the barrel (2010). If pressed against, for example as is when the menstrual collecting device is being released from the distal end (2010D) of the barrel (2010), the petal flaps (2011) are capable of assuming an open position in which the free edges of the petal flaps (2011) are moved away from each other, opening a way out of the barrel (2010).

One function of the petal flaps (2011) is to protect the user's tissues during insertion of the applicator (2000). Another function of the petal flaps (2011) is to protect the menstrual collecting device, e.g., (1000), from unfolding prematurely and before the applicator is fully placed by the user. During placement of the menstrual collecting device (10, 100, 200, 300, 400, 500, 600, 700, 800, 900 or 1000), the plunger (2012) may push the menstrual collecting device out of the barrel (2010). This pushing through-the-barrel motion opens the petal flaps (2011) away from the central axis of the barrel (2010), releasing, unfolding and placing the menstrual collection device under the cervix.

The barrels and/or plungers according to this disclosure may be made from mixed materials, for example, a plunger may be made from one blend of plastic materials, while the barrel may be made from another blend of plastic materials and/or the body of the barrel may be made one blend of plastic materials, while the petal flaps may be made of a different blend of plastic materials which are more flexible and bendable, allowing an easy opening of the petal flaps during a release of the menstrual collecting device from the applicator (2000).

In some embodiments, the entire length of the barrel (2010) from its distal end (2010D) to its proximal end (2010P) may be in the range from about 60 mm to about 120 mm, and more preferably from about 90 mm to about 100 mm. A length of the plunger (2012) correlates with the length of the barrel (2010). Preferably, when the plunger (2012) is fully inserted into the barrel (2010), the distal end (2012D) of the plunger (2012) may be aligned with the distal end (2010D) of the barrel (2010).

The length and width of the plunger (2012) may vary, but it is sized such that the plunger (2012) can be pushed through the barrel (2010), as shown in FIGS. 13B and 13C. A diameter of at least a portion of the plunger (2012) insertable into the barrel (2010) is smaller than a diameter of the hollow channel in the barrel (2010). In some embodiments, the length of the plunger (2012) may be from about 80 mm to about 140 mm, and more preferably from about 100 mm to about 120 mm.

In some embodiments, the plunger (2012) may contain one or more ridges (2014) located on the external surface of the plunger (2012) for at least a portion of the plunger length, the one or more ridges (2014) extending from the external surface of the plunger (2012). The one or more ridges (2014) function to reduce the friction between the plunger (2012) and the barrel (2010) when the plunger (2012) is pushed through the hollow channel of the barrel (2010), one embodiment of which is shown in FIG. 13B.

With reference to FIG. 14C, in some embodiments, the barrel (2010) according to this disclosure may contain a clicking mechanism which may include one or more rings (2020) protruding inside the barrel (2010) near its proximal end (2010P). The ring (2020) is attached to the internal surface of the barrel (2010). The purpose of the clicking mechanism is to regulate and/or monitor movement of the plunger (2012) inside the barrel (2010). In particular, the clicking mechanism may prevent the plunger (2012) from separating from the barrel (2010) after the menstrual collecting device has been placed and the user is withdrawing the applicator (2000).

With reference to FIG. 14A, in the embodiments, wherein the applicator (2000) contains a clicking mechanism, the plunger (2012) may further contain one or more notches (2022) on its external surface for at least over a portion of its length. The purpose of the one or more notches (2022), if present, is to work with the ring (2020), or in alternative with flanges/fingers instead of the ring (2020), aiding in the function of the clicking mechanism, preventing separation of the plunger (2012) from the barrel (2010) during withdrawal of the applicator (2000) after the menstrual collecting device has been placed.

When in use, the menstrual collecting device (10, 100, 200, 300, 400, 500, 600, 700, 800, 900 or 1000) may be folded and inserted into the applicator (2000 or 3010).

Thus, in yet further aspect, this disclosure relates to a system or kit that includes one or more menstrual collecting devices according to this disclosure and one or more applicators according to this disclosure, one embodiment, generally (2000) is shown in FIGS. 6A-6D.

In some embodiments, a menstrual collecting device may be pre-loaded and folded in the barrel of an applicator, one embodiment of which is shown in FIG. 6A, showing the menstrual collecting device (3016) being positioned in the barrel (3012) of the applicator (3010). Using an applicator supports/ensures a correct positioning of the menstrual collecting device (3016) under the cervix. The applicator (2000 or 3010) lowers the steep learning curve in menstrual collectors, e.g., menstrual cups and menstrual discs, which require messy manual folding and insertion of the device. The systems/kits may comprise one or more menstrual collecting devices and at least one applicator. The systems/kits may further include at least one user manual.

The menstrual collecting device and applicator according to this disclosure provide significant technical advantages and improvements over conventional menstrual cups. A conventional menstrual cup sits in the vaginal canal. In contrast, a menstrual collecting device according to this disclosure sits in the fornix, right below the cervix and can provide better leakage protection.

A system comprising one or more menstrual collecting devices according to this disclosure and at least one applicator according to this disclosure provide at least the following technical advantages. First, the applicator allows the menstrual collecting device to be easily inserted without mess from manual handling and places the menstrual collecting device properly. Second, the menstrual collecting device is long-lasting, preferably up to 12 hours. Third, the menstrual collecting device is comfortable. Fourth, it can be removed easily with the pull string or pull tab.

Toxic Shock Syndrome. The menstrual collecting devices according to this disclosure are collective and do not have absorption features, lowering the risk of TSS significantly.

Messy contact during insertion and removal. The applicator allows the users to avoid using their own fingers to insert the product. Furthermore, the pull string or the pull tab on the collecting device allows for easy removal without using their own fingers to locate and remove the product.

Difficulty in proper positioning. The applicator allows the user to push the plunger extravaginally for proper positioning in the fornix, solving the difficult manual positioning users currently must undergo, inserting their fingers intravaginally for proper positioning.

Difficulty in removal. The menstrual collecting device's attached pull string or pull tab allows the user to quickly and easily remove the product from the body with a simple tug of the pull string or pull tab.

The menstrual collecting devices and applicators according to this disclosure eliminate the difficult insertion and removal processes of menstrual cups and discs. The applicator minimizes challenges facing insertion while the menstrual collecting device's pull string or tab allows for easy, quick removal. In addition, the menstrual collecting device's return wall, if present, aids in retaining menses during and after use.

Methods for manufacturing products according to this disclosure may include injection molding, co-molding, heat bonding/sealing/staking, radiofrequency sealing, ultrasonic/thermal impulse/radiofrequency welding, dip molding, thermoforming, vacuum forming, compression molding, cutting, etc. The rim may include an expandable/contractible, adjustable feature, such as a co-molded ridged material to create a spring, opposing indents, a spiral molded with variable wall thicknesses, a spring coil molded with variable wall thicknesses, “C” spring, or any other suitable collapsing feature. The rim may be circular, elliptical, or any other suitable shape. Methods of manufacturing may include injection molding, co-molding, compression molding, etc. The collection bag may be thermoformed, cut, and/or heat staked in some embodiments to the rim, or it can be molded as a single injection part, etc. In some embodiments, the menstrual collecting product may be injection-molded in its entirety.

In further aspect, this disclosure relates to uses of the menstrual collecting devices and applicators according to this disclosure for personal care during a menstrual cycle. One embodiment of an application method, wherein the method comprises one or more of the following steps: folding and inserting the menstrual collecting device into the applicator barrel, inserting the plunger into the barrel, inserting the applicator barrel with the folded menstrual collecting device into the user's vagina, pushing the plunger of the applicator to release the menstrual collecting device and ensure its correct positioning under the cervix, removing the applicator, collecting menstrual discharge with the menstrual collecting device, and pulling the pull string or pull tab of the menstrual collecting device in order to remove the menstrual collecting device from the vagina after the use has been completed. In further embodiments, a kit may comprise of one or more menstrual collecting devices and at least one applicator. In these embodiments, the applicator may be preloaded with a folded menstrual collecting device, prior to the use for ease.

1. A menstrual collecting device comprising a collection bag having a rim, the collection bag created by a wall enclosing a chamber having a volume for collecting menstrual discharge, the collection bag having a length between a bottom surface and a front surface, the collection bag having an opening located on the front surface of the collection bag, the rim surrounding the perimeter of the opening and having a volume for collecting menstrual discharge, and a pull string or a pull tab being attached to the rim or the wall below the rim.

2. The menstrual collecting device of claim 1, wherein the menstrual collecting device is reusable or disposable.

3. The menstrual collecting device of claim 1, wherein the rim is defined further by one or more of the following features:
a) the rim is adjustable, and the adjustable rim includes an expandable/contractible feature,
b) the rim is a co-molded ridged material to create a spring, comprises opposing indents, is a spiral molded with variable wall thicknesses, is a spring coil molded with variable wall thicknesses, comprises a “C” spring, and/or comprises any other suitable collapsing feature;
c) the rim is substantially circular, elliptical, or any other suitable shape;
d) the rim is manufactured by a method which includes injection molding, co-molding and/or compression molding; and/or
e) the rim is concave.

4. The menstrual collecting device according to claim 1, wherein the menstrual collecting device further may comprise of a return wall positioned over a portion of the opening of the menstrual collecting device, the return wall being attached to the rim and/or to the wall of the menstrual collecting device.

5. The menstrual collecting device according to claim 4, wherein the return wall is defined further by one or more of the following features:
a) the return wall is angled downwards transitioning into the opening;
b) the opening is located at the center or side of the rim;
c) the opening takes the shape of a circle, a hexagon, a water droplet, a slit, an “x”, or any other suitable shape for collection of menstrual discharge in the collection bag;
d) the return wall's function is to withhold collected vaginal discharges or menses from escaping; and/or
e) the opening can be closed or sealed through different mechanisms involving folding, sealing, and/or covering the return wall for ease of disposal.

6. The menstrual collecting device of claim 1, wherein the collection bag is defined further by one or more of the following features:
a) some or all parts of the collection bag have different wall thickness to prevent sudden spillage upon the user's activity;
b) the collection bag is contoured to prevent spillage; and/or
c) the collection bag is in the shape of a semi-sphere, a spherical ellipsoid, the frustum of a cone, or a semi sphere that is flattened near the bottom.

7. The menstrual collecting device of claim 1, wherein the pull string or a pull tab is defined further by one or more of the following features:
a) the pull string is connected to the menstrual collecting device on or below the rim and serves to ease removal for the user;
b) the pull string or the pull tab is connected to the return wall;
c) the pull string or the pull tab may have texture for the user's grip; and/or
d) the pull string or the pull tab is wider at its point of attachment to the menstrual collecting device and tapers out.

8. An applicator composed of a barrel and a plunger, the plunger being insertable into the barrel, the barrel sized for housing the menstrual collecting device according to claim 1 in a folded form and for inserting the menstrual collecting device under the cervix by pushing the plunger.

9. The applicator of claim 8, wherein the applicator is reusable or disposable.

10. The applicator according to claim 8, wherein the applicator is further defined by one or more of the following features:
a) the barrel is straight, curved or angled;
b) the plunger is sized and shaped to mirror the shape of the barrel, and push a menstrual collecting device out of the applicator barrel and into the fornix;
c) the applicator has a clicking mechanism having a ring or flanges/fingers protruding inside the barrel and a portion of the external surface of the plunger having one or more notches;
d) the barrel contains two or more petal flaps at its distal end;
e) the barrel contains one or more grooves or ridges on the barrel wall for at least a portion of the barrel length, the grooves or ridges being located opposite or substantially opposite to each other;
f) the barrel is made of a material different than the plunger;
g) the petal flaps are made of a material which is softer and more flexible than the rest of the barrel body;
h) the plunger is hollow;
i) at its proximal end, the plunger ends with a stopper; and/or
j) the plunger contains one or more ridges located on the external surface of the plunger for at least a portion of the plunger length, the one or more ridges extending from the external surface of the plunger.

11. A system or a kit comprising at least one applicator of claim 8 and one or more of the menstrual collecting devices comprising a collection bag having a rim, the collection bag created by a wall enclosing a chamber having a volume for collecting menstrual discharge, the collection bag having a length between a bottom surface and a front surface, the collection bag having an opening located on the front surface of the collection bag, the rim surrounding the perimeter of the opening and having a volume for collecting menstrual discharge, and a pull string or a pull tab being attached to the rim or the wall below the rim.

12. A method for manufacturing the menstrual collecting device, applicator or the system according to this disclosure, wherein the method includes one or more of the following: injection molding, co-molding, heat bonding/sealing/staking, radiofrequency sealing, ultrasonic/thermal impulse/radiofrequency welding, dip molding, thermoforming, forming, compression molding, and/or cutting.

13. A method for collecting menstrual discharge, the method comprising:
folding and inserting the menstrual collecting device comprising a collection bag having a rim, the collection bag created by a wall enclosing a chamber having a volume for collecting menstrual discharge, the collection bag having a length between a bottom surface and a front surface, the collection bag having an opening located on the front surface of the collection bag, the rim surrounding the perimeter of the opening and having a volume for collecting menstrual discharge, and a pull string or a pull tab being attached to the rim or the wall below the rim into the applicator barrel of the applicator according to claim 8,
inserting the plunger into the barrel,
inserting the applicator barrel into the user's vagina,
pushing the plunger of the applicator to release the menstrual collecting device and ensure its correct positioning under the cervix,
removing the applicator,
collecting menstrual discharge with the menstrual collecting device, and
pulling the pull string or the pull tab of the menstrual collecting device in order to remove the menstrual collecting device from the vagina.
